(19) United States
(12) Patent Application Publication
(10) Pub. No.: US 2026/0118272 A1
(43) Pub. Date: Apr. 30, 2026

(54) ZERO-POWER, OPTICAL TOXIC GAS AND VAPOR SENSORS UTILIZING PRINTED NEMATIC LIQUID CRYSTAL PATTERNS ON SELECTIVELY REACTIVE SUBSTRATES

(71) Applicant: Kent State University, Kent, OH (US)

(72) Inventors: Marianne Estelle Prévôt, Kent, OH (US); Grace Ann Rohaley, Kent, OH (US); Ryan Williams, Kent, OH (US); Torsten Hegmann, Kent, OH (US)

(21) Appl. No.: 19/370,969

(22) Filed: Oct. 28, 2025

Related U.S. Application Data
(60) Provisional application No. 63/712,623, filed on Oct. 28, 2024.

(51) Int. Cl. G01N 21/77; G01N 33/00

(52) U.S. Cl. CPC G01N 21/77; G01N 33/0047; G01N 33/0052; G01N 2021/7783

(57) ABSTRACT

A sensor includes, in sequence: a first polarizer having a first polarization direction; a first transparent substrate; optionally an alignment underlayer; a reactive layer comprising: nanoparticles substantially covered by one or more ligands capable of selectively, chemically reacting with one or more gases; or a self-assembled monolayer capable of selectively, chemically reacting with one or more gases; an inert nematic liquid crystal layer patterned on the reactive layer; a second transparent substrate; and a second polarizer having a second polarization direction perpendicular to the first polarization direction.

This application claims priority to U.S. Provisional Application Ser. No. 63/712,623 filed Oct. 28, 2024, the contents of which is incorporated by reference herein.

### STATEMENT REGARDING FEDERALLY SPONSORED RESEARCH

The invention was made with government support under Grant No. PFI-RP-2122421 awarded by the National Science Foundation. The government has certain rights in this invention.

### BACKGROUND

Professions such as firefighters, military personnel, and other first responders are frequently exposed to dangerous situations where they may come into contact with cancer-causing toxins or harmful chemical gases that pose significant health and safety risks. When emergency personnel attend to accidents, fires, or disasters, they often do so without full awareness of the potentially harmful or deadly chemicals. For example, research and atmospheric testing at fire scenes have revealed that toxic fumes are present at all stages of a fire and can persist at high levels even after the fire has been extinguished. Proper site surveys and thorough analysis can help protect or mitigate these hazardous situations using personal protective equipment (PPE) and gear. However, market research has revealed that emergency personnel often lack the time for a comprehensive site investigation in life-or-death situations. This means they cannot be sure if the air is safe to breathe—a potentially fatal uncertainty. Similarly, when dealing with chemical warfare agents or chemical leaks in manufacturing facilities, it is crucial to evaluate the severity of the hazards in order to determine the level of contamination. This assessment will help advise first responders and military personnel on the appropriate PPE for the given situation.

These situations illustrate the critical need to actively monitor for immediate and long-term exposures to toxic gases and vapors for first responders. Available commercial gas sensor equipment ranges from small, wearable units to large, heavy, and difficult-to-use devices. Smaller, handheld detectors can often only detect a single gas or a small number of gases. These include gas detection tubes or badges. In contrast, bulkier gas detectors can sample, detect, and identify a wide range of hazardous gases. These mainstream sensors use electronic micro-processing to convert the signal to a display output signal. This means, the technology relies on electrical power to operate and show images or text. To increase safety and to equip personnel in remote areas such as wildland fires, zero-power optical sensors would provide an additional layer of safety or, in some cases, the only feasible safety solution. Furthermore, interviews were conducted with both urban and wildland firefighters indicated that toxic gas sensor design needs to be intricate enough to outfit all personnel directly (i.e., each and every member of a team), be simple to read, easy to operate, and low-cost.

A multi-mode liquid crystal (LC) sensor platform that requires zero power to operate and can, based simply on the device design, be used as ppt-level to analytical ppm-level (dose×time) sensors was disclosed by Applicant, for example in U.S. Patent Pub. No. 2020/0355664 A1, which is incorporated by reference herein in its entirety. Inkjet printing of nanoparticles with a reactive ligand shells that affect the anchoring of nematic LC (N-LC) molecules facilitated the creation of sensors devices that produced an unmistakable warning or image solely based on the transmission or reflection of light. This was particularly relevant since potentially competitive gas sensor badge technologies in the market are analytically accumulative but require the observer to distinguish color and color intensities. One or several forms of color vision deficiency (color blindness) affects about one in 12 men and one in 200 women, and men represent the majority among emergency responders, military personnel, firefighters, and members of HAZMAT teams. Sensors exclusively based on a colorimetric readout could impair or disable the wearer in emergency situations due to color perception changes such as caused by other spectral stimuli, seasonal changes, age, or differences between daytime and nighttime. In discussions with fire department chiefs and firefighters, numerous communicated their color vision deficiency to Applicant.

The design and operation of the previously reported sensors provided solutions for the detection of chlorine, phosgene, and aliphatic amines. The sensing mechanism was composed of two essential and interdependent elements. The first element is a reactive material based on monolayer-capped nanoparticles (NPs), which interact sensitively and selectively with toxic target gases or vapors using selective surface chemistries on the NP surface. A chemical reaction modifies, irreversibly, the nanoparticles' surface functional groups or oxidizes the ligand shell (followed by desorption from the NP surface). To observe this modulation with the naked eye, a material capable of showing these otherwise often imperceptible changes was used. Since the anchoring conditions of N-LCs are highly sensitive to intermolecular interactions at surfaces, LCs were used to generate the visual responses via molecular reorientation. Therefore, the second element of the design was a N-LC layer whose anchoring is sensitive to the NP's surface chemistry changes that, in turn, serves as a reconfigurable alignment layer, just as commonly used in LC display devices using an applied electric field. Thus, the N-LC acts as a signal amplifier via molecular orientational changes that are visualized by sandwiching the device between crossed polarizers.

For example for chlorine gas (Cl₂), the LC molecular orientation is initially perpendicular to the substrate, which corresponds to no light transmission. Upon exposure to the targeted gas, the localized molecular orientation is irreversibly disturbed (from vertical to horizontal orientation). By patterning (inkjet printing) the reactive gold NPs (GNPs) on substrates with an additional polymer-based alignment layer, the resulting light transmission contrast can be used generate text, images, or whatever design (motif) the user desires. This sensor principle enabled the creation of a sensor device platform that produces unmistakable visual warnings solely based on light transmission and light blockage (a in FIG. 1). However, the response time and sensitivity of these sensor devices was governed by the diffusivity of the targeted toxic gases through the ˜5-micron thin N-LC layer. To reach the reactive, patterned GNP layer, the gas or vapor has to diffuse through about 5 mm or more of the N-LC layer before reaching the reactive GNPs, and the patterned N-LC anchoring transition gradually proceeded from the outside to the center of the GNP pattern. With diffusivity values of gases or vapors through N-LCs of ˜1×10⁻⁶ cm² s⁻¹ (i.e., ˜100 μm s⁻¹), the fastest possible response times for the edge of the pattern to change the anchoring of the N-LC was about 8.3 min. While this diffusivity-controlled mode lends itself perfectly to the construction of dose×time sensors, i.e., gradually turning on a pattern over time, immediate response sensors for acute levels of gas toxicity set in the US by the Occupational Safety and Health Administration (OSHA) and the National Institute for Occupational Safety and Health (NIOSH) required a complete redesign of this sensor platform that drastically reduces the path of the toxic gas or vapor through the N-LC to reach the reactive layer that in turn alters N-LC anchoring.

### BRIEF DESCRIPTION

The present disclosure relates to systems and methods for detecting gases (e.g., hazardous gases) which utilize patterned nematic liquid crystal droplets on a reactive species (surface functionalized nanoparticles or self-assembled monolayers capable of reacting with one or more target gases).

Disclosed, in some embodiments, is a sensor including, in sequence: a first polarizer having a first polarization direction; a first transparent substrate; optionally an alignment underlayer; a reactive layer containing nanoparticles substantially covered by one or more ligands capable of selectively reacting with one or more gases or a self-assembled monolayer capable of selectively reacting with one or more gases; an inert nematic liquid crystal layer patterned on the reactive layer; a second transparent substrate; and a second polarizer having a second polarization direction perpendicular to the first polarization direction. The sensor may further include an alignment underlayer between the first transparent substrate and the reactive layer. In some embodiments, the alignment underlayer contains polyimide or SiOₓ. The nanoparticles may be metallic nanoparticles, such as gold, silver, platinum or palladium nanoparticles. The reactive layer may include aliphatic thiol-capped gold nanoparticles. In some embodiments, the one or more gases includes chlorine (Cl₂) gas or phosgene (COCl₂) gas. The self-assembled monolayer may be a siloxane-based self-assembled monolayer. In some embodiments, the reactive layer includes (3-aminopropyl) trialkylsilane (e.g., (3-aminopropyl) triethylsilane or (3-aminopropyl)trimethylsilane). The nematic liquid crystal layer may be doped with a mesogenic carboxylic acid (e.g., 4-hexylbenzoic acid).

Disclosed, in other embodiments, is a method of determining whether a target gas is present in an environment. The method includes providing a sensor to the environment; and visually inspecting the sensor for a change indicating the target gas is present. The sensor includes a first polarizer having a first polarization direction; a first transparent substrate; optionally an alignment underlayer; a reactive layer containing nanoparticles substantially covered by one or more ligands capable of selectively reacting with the target gas or a self-assembled monolayer capable of selectively reacting with the target gas; an inert nematic liquid crystal layer patterned on the reactive layer; a second transparent substrate; and a second polarizer having a second polarization direction perpendicular to the first polarization direction.

Disclosed, in further embodiments, is a method of forming a gas sensor. The method includes ink-jet printing nematic liquid crystal droplets onto a reactive species. The reactive species includes surface functionalized nanoparticles or silane-monolayers capable of reacting with one or more target gases.

These and other non-limiting aspects of the disclosure are more particularly set forth below.

### BRIEF DESCRIPTION OF THE DRAWINGS

The following is a brief description of the drawings, which are presented for the purposes of illustrating the exemplary embodiments disclosed herein and not for the purposes of limiting the same.

FIG. 1: a) Earlier zero-power LC sensor platform design utilizing printed GNPs and sandwiched N-LC films between two substrates. This design favored the use for dose×time sensors given the dependency of the response time to the diffusivity of the gas through the N-LC layer. b) Revised design based on printed N-LC pattern atop reactive layers of GNPs or SAMs providing immediate access of the gas to the reactive layer through small printed and patterned N-LC droplets. 3-D renderings show principal device designs (overall working principle and annotated side views) as well as examples of actual devices before, after, or during exposure to Cl₂ gas.

FIG. 2: a) Bright field microscope image of N-LC (MLC-2169) droplets inkjet printed on a polyimide alignment layer with a drop spacing of ˜100 μm (center-to-center), b) bright field microscopy image of N-LC printed atop dense DDT-GNP layer (drop spacing=90 μm center-to-center), c) polarized optical photomicrograph (crossed polarizers) of the N-LC printed on a rubbed polyimide alignment layer inducing planar anchoring (top-right section of the image; likely leading to a radial hedgehog-type configuration: i.e., an s=+1 defect escaping into the third dimensions with the homeotropic boundary conditions at the opposing N-LC/air interface) and homeotropic anchoring at both confining interfaces when the N-LC is printed on DDT-GNPs (lower left section of the image), d) bright field microscopy images of the N-LC printed on lines of DDT-GNPs generated by overlapping of printed GNP in o-xylene droplets (both after drying with drop spacing=90 μm, center-to-center), e) microphotograph taken by tilting a final substrate with the N-LC printed on DDT-GNPs. The enlarged section (inset) clearly shows that the N-LC droplets are flat, pancake or coin-like (3-D rendering in inset), and f) microphotograph taken of a printed substrate after breaking it in half, showing that both the individual N-LC droplets as well as the pattern itself stay intact after mechanical impact.

FIG. 3: a) Flowchart of the experimental setup for Cl₂ gas testing, b) photographs of the setup showing the two mass flow controllers (MFCs) on the fume hood counter (left image) as well as the digital microscope and testing chamber (right image), c) photographs of a few examples of possible pattern designs, d) snapshots of a prototype test video with 50 ppm Cl₂ gas (time stamps left-to-right are: 0, 30, 70, 90, and 94 s, when no further change was observed), e) response of sensor fabricated with DDT-GNPs on homeotropic polyimide using MLC-2169 after exposure to 10 ppm Cl₂ gas, and f) photomicrograph of patterned N-LC droplet without (left image) and with crossed polarizers (right image) before exposure.

FIG. 4: Prototype testing of devices with simple printed rectangular N-LC pattern tested at increasingly lower concentration of Cl₂ gas as well as using two different N-LCs: a) with printed MLC-2169 and b) printed 5 CB (scale bar for all images=1 cm).

FIG. 5: Plot of the average sensor response time [s] vs. Cl₂ gas concentration [ppm] for the prototype devices using MLC-2169 tested using the setup shown in FIG. 3; lower curve: onset of recognized change in light transmission; upper curve: complete change in light transmission (no further change noticeable). Data acquired by analyzing changes in light transmission using the greyscale image changes in the active device area from the video snapshots using ImageJ®; OSHA: IDHL=immediately dangerous to life and health; Emergency Response Planning Committee of the American Industrial Hygiene Association: ERPG 3=emergency response planning guidelines-maximum airborne concentration below which it is believed that nearly all individuals could be exposed for up to one hour without experiencing or developing life-threatening health effects.

FIG. 6: Water submersion test of prototype devices: a) after exposure to Cl₂ gas (tripped sensor) and b) prior to exposure to Cl₂ gas (non-tripped sensor).

FIG. 7: a) Flowchart of the experimental setup for Cl₂ gas testing under the additional influence of dense, in situ generated smoke, b) photographs of the setup showing smoke generation (left) and smoke-filled testing chamber (right), c) photographs of snapshots of a prototype test video with 50 ppm Cl₂ gas and additional two bursts of dense smoke seen in the images 3 and 5 from the left (scale bar=1 cm), and d) photographs of the luminescent carbon dots isolated from the smoke generated by burning word chips (exposed to UV light at λₑₓ=365 nm).

FIG. 8: a) Principle of detection of COCl₂ using silane-modified alignment layers on glass substrates via the addition of 6 BA to the N-LC, b) snapshots taken during the detection of 7.5 ppm COCl₂ (top) and photomicrographs (10° decrossed polarizers) before and after COCl₂ exposure, c) snapshots taken during the detection of 660 ppb COCl₂ (time-stamped), and d) during the detection of 300 ppb COCl₂.

FIG. 9: Plot of the average sensor response time [s] vs. COCl₂ gas concentration [ppm] for the prototype devices using MLC-2169+3 wt % 6 BA tested using the setup shown in FIG. 3; clearly discernable onset of recognized change in light transmission; NIOSH: IDHL=immediately dangerous to life and health; Emergency Response Planning Committee of the American Industrial Hygiene Association: ERPG 3=emergency response planning guidelines−maximum airborne concentration below which it is believed that nearly all individuals could be exposed for up to one hour without experiencing or developing life-threatening health effects; EEGL=emergency exposure guidance level.

FIG. 10: a) Firefighters equipped with sensor prototypes while conducting training, b) carabiner attachment for helmets and turnout gear, and c) types of sensor designs provided to the firefighters. Responses by firefighters collected after training regarding d) overall device usefulness, e) form factor and image visibility, and f) possible further (necessary) prototype improvements.

FIG. 11 is a cross-sectional view of a non-limiting embodiment of a sensor in accordance with some embodiments of the present disclosure.

FIG. 12 is a cross-sectional view of another non-limiting embodiment of a sensor in accordance with some embodiments of the present disclosure.

### DETAILED DESCRIPTION

The present disclosure may be understood more readily by reference to the following detailed description of desired embodiments and the examples included therein. In the following specification and the claims which follow, reference will be made to a number of terms which shall be defined to have the following meanings.

Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

The term “comprising” is used herein as requiring the presence of the named components/steps and allowing the presence of other components/steps. The term “comprising” should be construed to include the term “consisting of”, which allows the presence of only the named components/steps.

Numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values). The endpoints of the ranges and any values disclosed herein are not limited to the precise range or value; they are sufficiently imprecise to include values approximating these ranges and/or values.

As used herein, the term “about” when used in conjunction with a stated numerical value or range denotes somewhat more or somewhat less than the stated value or range, to within a range of ±10% of that stated.

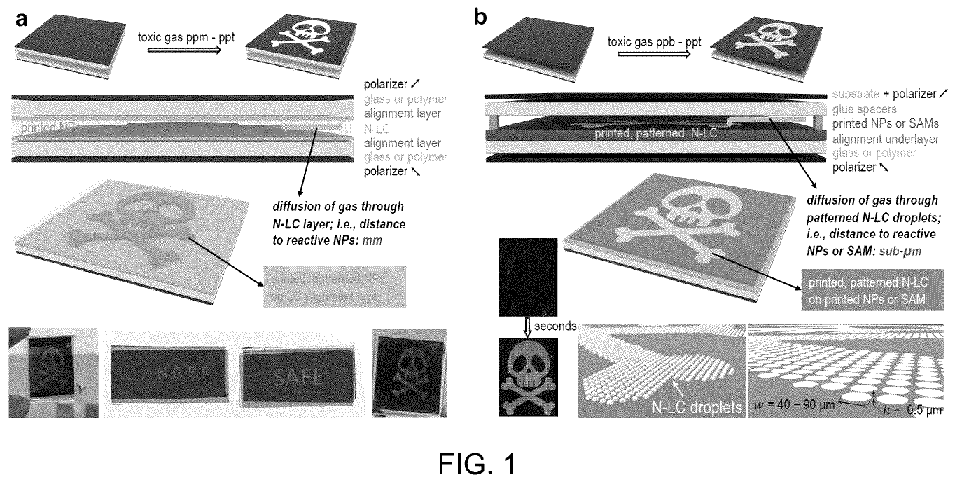

The present disclosure relates to systems and methods for sensing toxic gases by utilizing printed nematic liquid crystal patterns. As depicted in FIG. 1 (b), the sensor generally includes, in sequence, a first polarizer, a first transparent layer, an alignment underlayer, a reactive layer comprising nanoparticles or SAMs, patterned nematic liquid crystal (N-LC) droplets, a second transparent layer, and a second polarizer. One or more spacers (e.g., glue spaces) may be included to affix the second transparent layer which protects the reactive layer. Gas can diffuse through the patterned N-LC droplets to the reactive layer.

### Reactive Layer

The reactive layer may be formed from ligand-bound nanoparticles or a self-assembled monolayer.

The nanoparticles may be metal nanoparticles, such as gold, silver, platinum, or palladium nanoparticles. The nanoparticles may alternatively or additionally include non-metallic nanoparticles, such as carbon dots. The average diameter of the nanoparticles may be in the range of from about 1 nm to about 20 nm, including from about 1 nm to about 10 nm. Specific types of ligands adhere to the nanoparticle surface by a chemical bond; either coordinative bonding, ligand to metal coordination, or covalent bonding. A key aspect in the selection of various ligands to detect specific gases such as halogens, phosgene, etc. is that they do not chemically react with the associated gas, which is to be detected. The one or more ligands at least partially or substantially cover the one or more nanoparticles, as for example at least about 60%, or about 80%; desirably at least about 90%, and preferably at least about 95%; or the entire (i.e. total) nanoparticle surface area. Ligands are selected by specific chemical reactions that result in a change in surface environment at the interface to the liquid crystal molecules. This change in surface environment leads to a change in liquid crystal alignment and as a consequence a change in light transmission between crossed polarizers. Non-hazardous gases and vapors include acetone, other ketones, and the like. Examples of hazardous gases include halogens comprising chlorine, bromine, or iodine, cyanide such as hydrogen cyanide, phosgene, aliphatic amines, dimethyl sulfide and dimethyl selenium, hydrazine, or non-hazardous gases such as ketones including chalcogenides, or any combination thereof.

A more detailed discussion of particular ligands can be found in U.S. Patent Pub. No. 2020/0355664 A1, which is incorporated by reference herein in its entirety.

### Patterned Nematic Liquid Crystal Layer

The nematic liquid crystal layer is ink-jet printed or otherwise patterned onto the reactive layer. The pattern may be intended to provide a clear visual signal upon detection of one or more target gases. For example, the pattern may be a skull-and-crossbones as depicted in FIG. 1.

Center-to-center distances between adjacent droplet may be in a range of from about 20 μm to about to about 250 μm, including from about 60 μm to about 120 μm or about 90 μm.

The droplets may have volumes in a range of from about 0.1 to about 10 pL, including from about 1.5 pL to about 4 pL or about 2.4 pL.

### Alignment Underlayer

The alignment layer is provided on the first transparent layer and functions as an adhesion layer for the reactive layer. Non-limiting examples of suitable materials for the alignment underlayer include polyimide, silicon oxides (SiOₓ, 0<x≤2), and polyvinyl alcohol. The alignment layer may have a thickness in a range of from about 50 Å to about 500 Å, including from about 50 Å to about 250 Å, or about 50 Å to about 150 Å.

The alignment underlayer may be optional in some embodiments. For example, the reactive layer may include a self-assembled monolayer (e.g., a siloxane-based self-assembled monolayer) formed directly on the first substrate without the inclusion of an alignment underlayer.

### Transparent Layers

The transparent layers may be made from the same or different materials. Non-limiting examples of suitable materials include glass and transparent polymers. Non-limiting examples of transparent polymers include polycarbonate, poly(methyl methacrylate), or polyethylene terephthalate, among others. Each transparent layer may independently have a thickness in a range of from about 0.5 mm to about 2 mm, including from about 0.5 to about 1.5 mm, or about 1 mm to about 2 mm.

The first and second polarizers may be made from the same or different materials. Each polarizer may be in the form of a film laminated to its respective transparent layer. The polarizers are provided for cross-polarization, meaning that the polarization direction of the first polarizer is perpendicular to the polarization direction of the second polarizer.

### Sensor Design for Immediate, Acute Exposure

In contrast to the previous design (a in FIG. 1), the new sensor design concept, depicted in FIG. 1 (b), now features only one substrate (glass or potentially also polymer-based) is treated with an alignment underlayer (SiOₓ or polyimide) onto which the reactive GNPs are printed, dense enough to cover almost the entire substrate (˜2×3 cm). When using reactive GNPs, the alignment underlayer serves as an adhesion layer for the printed aliphatic thiol monolayer-capped GNPs. The other side of this substrate is laminated with a linear polarizer film. A chemically inert N-LC or N-LC mixture is then patterned in any desirable design by inkjet printing atop the GNP layer. Using sufficiently high spacer glue or spacer tape in the corners or along the edges of the device, a second substrate is affixed to simply protect the printed N-LC pattern. This second substrate is also laminated with a linear polarizer, crossed with the first one (cross-polarization). In this design, the toxic gas or vapor has immediate, full access to the entire pattern of printed N-LC droplets, whose volume and thus, diameter and height can be controlled by setting the parameters of the ink-jet printing process accordingly. Since N-LCs favor homeotropic (vertical) anchoring at air interfaces, the reactive GNP surface chemistry also needs to induce homeotropic anchoring before toxic gas exposure to generate a non-translucent black ground state device. As soon as the chemical reaction takes place at the GNP surface (e.g., by oxidation and desorption of aliphatic thiol monolayers on GNP surfaces by Cl₂ gas), the N-LC droplets atop the GNP layers experience the desired anchoring transition from homeotropic to planar. The ensuing overall hybrid anchoring conditions then reveals a translucent image of the printed, patterned N-LC droplets. This design also opened the door for another device simplification. Since the reactive species is no longer patterned, selectively reactive self-assembled monolayers (SAMs), which can simply be grafted onto glass or polymer substrates, can now be used as homeotropic alignment layers.

### Feasibility of N-LC Printing

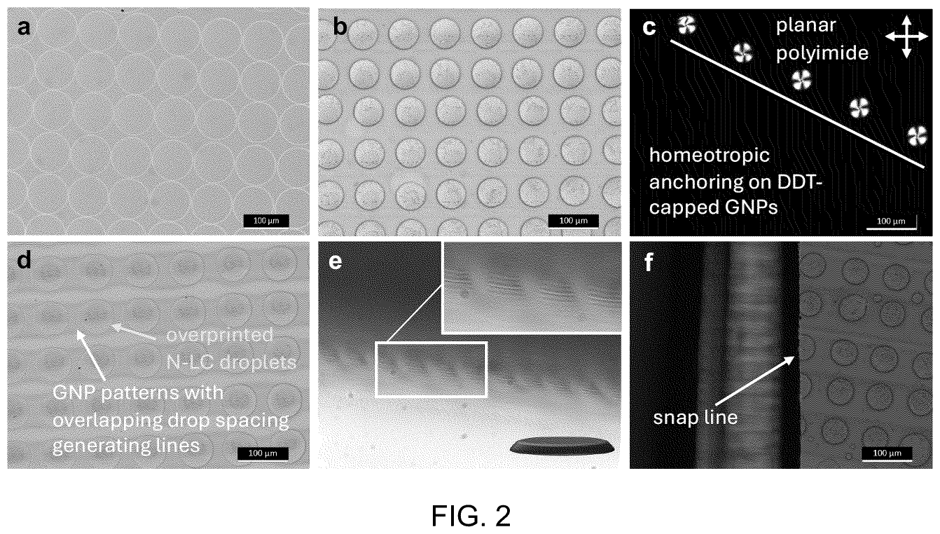

To ensure the final devices can withstand the anticipated working conditions and environments, different printing conditions for the N-LC patterns were investigated. To do so, a commercial N-LC mixture (MLC-2169, Merck) was printed on glass substrates coated with homeotropic polyimide or SiOₓ alignment layers with an initial drop spacing of 100 μm (center-to-center) and drop volumes, either 10 pL or 2.4 pL (a in FIG. 2). SiOₓ alignment layers provide additional chemical inertness to these devices.

Furthermore, since the deposition angle can be adjusted, N-LC anchoring can be tuned to allow for additional device iterations (e.g., a translucent surrounding when text is the active area). Adjustments in drop volume and spacing, further affected by the wetting of the N-LC on the required dodecanethiol (DDT)-capped GNP printed substrates allowed a determination that a drop spacing of 90 μm for the printed N-LC and drop volumes of 2.4 pL led to optimal printing conditions (b in FIG. 2) where the N-LC droplets did not coalesce at elevated temperatures, given that the final devices would potentially be exposed to higher temperatures in fire scenarios. The next test to be conducted was to print the N-LC atop a prior printed layer of DDT-capped GNPs to ascertain that the droplets would assume homeotropic anchoring at both interfaces, the DDT-GNPs on one substrate as well as air at the other (b in FIG. 2). The N-LC can effectively be printed on GNP multilayers as well as atop printed GNPs with sufficient overlap to generate GNP lines (c and d in FIG. 2). Tilting of such printed N-LC pattern also allowed visualization that the N-LC droplets are rather flat and pancake-like when experiencing the two confining substrates, DDT-GNPs and air (e in FIG. 2). Finally, the ruggedness of these printed substrates was tested by breaking one along the center of the print or dropping them on hard surfaces from a certain height. As shown in f in FIG. 2, even individual N-LC printed islets are not destroyed and continue to adhere to the substrate at the snap line.

### Cl₂ Gas Sensors: Testing and Sensor Refinements

Prototypes based on the new device design using already established alkylthiol-capped GNPs chemistry to detect Cl₂ gas were tested. Devices were constructed, as outlined in more detail below, by printing freshly synthesized (or properly stored) DDT-GNPs in o-xylene as a carrier fluid atop glass substrates coated either with a polyimide alignment layer favoring homeotropic anchoring and supporting GNP adhesion or SiOₓ deposited by thermal evaporation at varying angles to adjust N-LC anchoring and GNP adhesion conditions. The N-LC was then printed on top of the DDT-GNP layer, and the devices assembled as shown in b in FIG. 1.

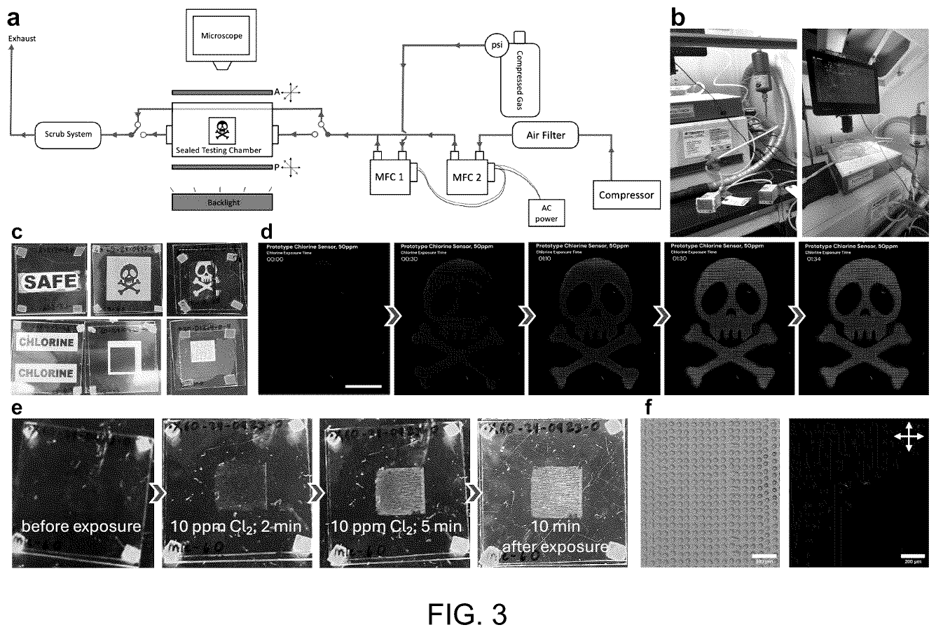

The testing setup is schematically shown in FIG. 3 in a (a photograph of this setup in b). Using commercially available pre-calibrated Cl₂ gas and utilizing mass flow controllers (MFC), practically any relevant concentration of Cl₂ gas could be tested. As stated before, the new device design with proper adjustment of alignment underlayers and printing conditions of both components allows for a multitude of designs of images, geometric figures, or text appearing or disappearing (e.g., the word “SAFE”; c in FIG. 3). An example of a sensor tested at 50 ppm Cl₂ started to visually trip, now after only 30 s, with a very clear skull and crossbones image visible after about 90 s (d in FIG. 3). Careful adjustments and continuous improvements of printing parameters now allows detection of even lower ppm levels of Cl₂ gas in seconds (e and f in FIG. 3). This includes levels of Cl₂ gas down to 10 ppm and less, which are relevant levels of exposure set by OSHA and NIOSH (the immediately dangerous to life and health (IDLH) value is 10 ppm for Cl₂). Notably, as could be seen in the snapshots from videos taken, the anchoring transitions now occur practically everywhere on the device at the same time and not, as before, gradually from the outside towards the center of the device.

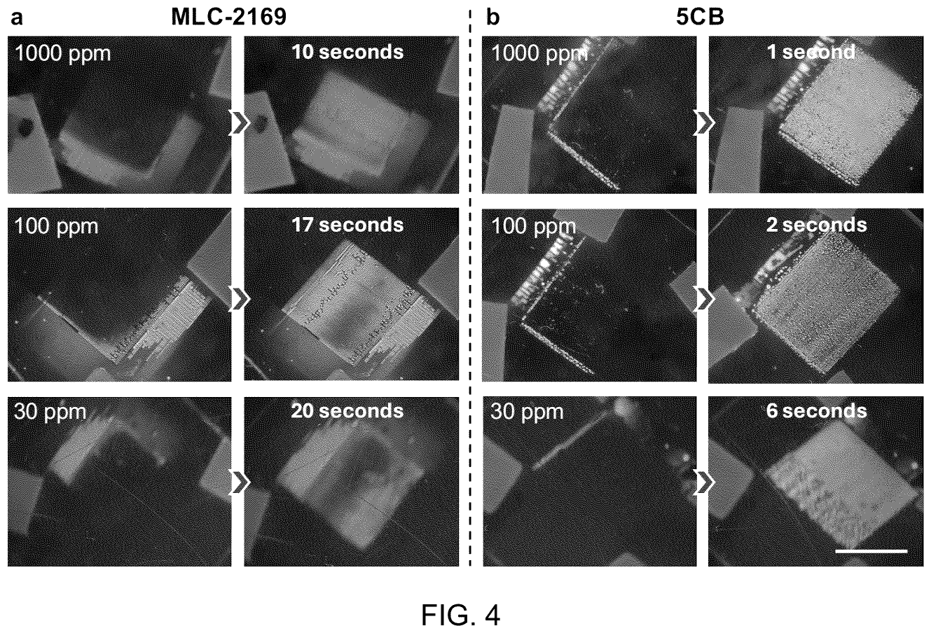

Another crucially important aspect and finding is that the new device concept gives access to toxic gas concentration-dependent response times as shown in FIG. 4.

Furthermore, different types of N-LCs give rise to faster or slower response times.

Notably, acute high concentrations of Cl₂ gas (˜1,000 ppm) now generate response times of 10 s or less: even only 1 s when using 5 CB. At OSHA/NIOSH critically relevant concentrations of 30 ppm (that would otherwise lead to immediate substernal chest pain, shortness of breath, and cough), response times are now 20 s or even only 6 s (b in FIG. 4). This faster response time of the sensors may be attributed to using printed 5 CB to the lower viscosity of 5 CB at room temperature, i.e., the testing temperature of T˜22° (ηMLC-2169=42 mPa·s vs. η5 CB=28 mPa·s). Thus, given the achieved sensitivity and response times, members of first responder, HAZMAT or military teams or anyone exposed to Cl₂ gas would know quickly and easily to either evacuate or if it was safe to remove safety gear such as a self-containing breathing apparatus (SCBA).

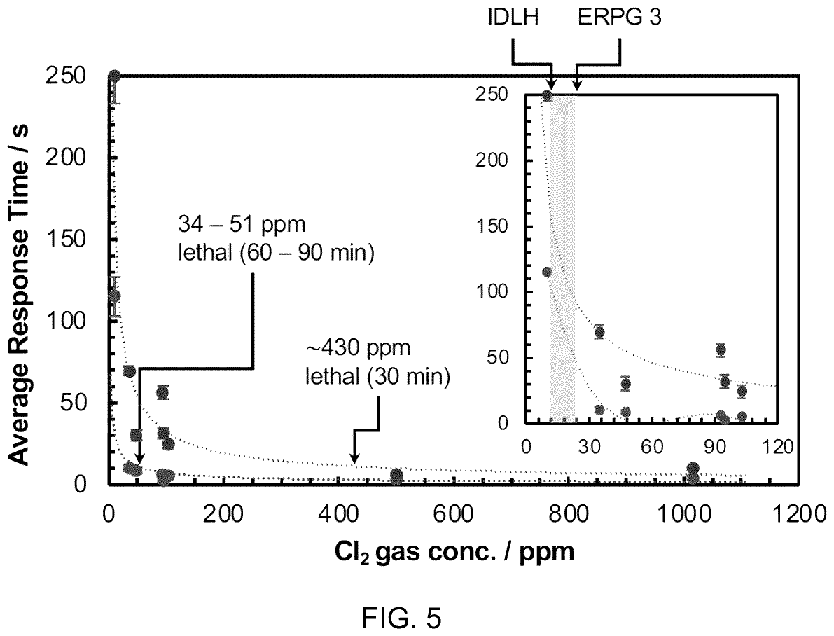

Performing these concentration-dependent tests over a wider range of concentrations further allowed the relationship between Cl₂ gas exposure levels and easily perceived sensor trip (or response) time to be examined (FIG. 5). Taking the human element out of recognizing the appearances of the anchoring transition patterns (onset of a clearly visible change in light transmission as well as full change in light transmission), ImageJ® was used to characterize the change in light transmission using changes in greyscale in the snapshots taken from the device testing videos, corresponding to 50% (onset) and 90% (complete change) of change in perceived brightness.

The plots of the sensor response time vs. Cl₂ gas concentrations demonstrate that the current sensor design is suitable at critically relevant levels of exposure with response times (i.e. noticeable changes in light transmission) of only tens of seconds or just under 2 minutes.

### Interference Testing: Smoke and Immersion in Water

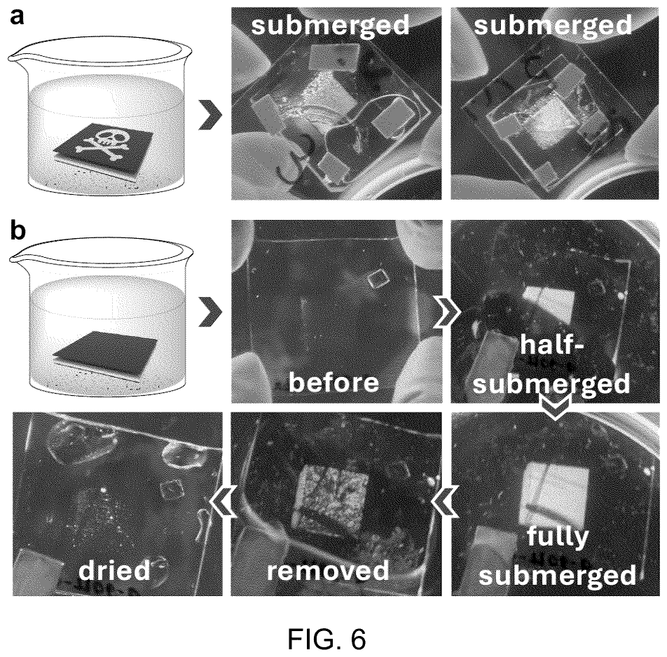

Given that these devices are intended for the use by emergency response personnel (e.g., firefighters), the next set of tests was geared towards assessing how these prototypes would behave in close to real world scenarios. In the first set of tests, these devices were subjected to water, taking the worst-case scenario of simply drowning the devices under water (FIG. 6). In the first set of tests, devices that had already tripped after exposure to low ppm levels of Cl₂ gas were submerged (a in FIG. 6). The final overall hybrid anchoring after exposure to Cl₂ is not disturbed since the top interface to air, now exposed to water, flips from homeotropic to planar anchoring as water replaces air, e.g., once tripped, the result would not be affected even if the device was fully submerged. However, a non-tripped device prior to Cl₂ gas exposure and going through that exact same top interface anchoring transition would appear as a false positive (i.e., the device appears tripped by simple exposure to water). Favorably, after drying the devices in air, the original homeotropic anchoring is restored and the device ready for sensing. To eliminate this issue, the final carrying containers for the devices (vide infra) or even just the edges can be treated with hydrophobic or even omniphobic coatings to fully prevent any kind of water or liquid penetration into the active area.

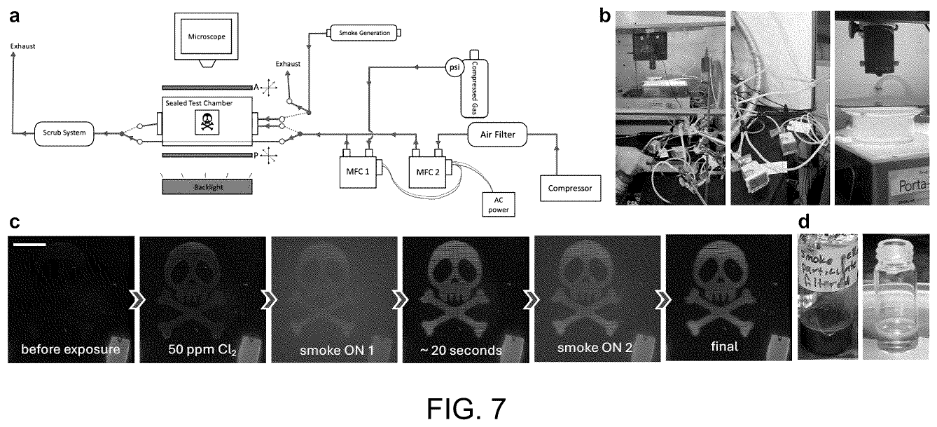

If, as envisioned, these sensors are used in fire situation, they would also need to withstand harsh conditions such as exposure to smoke. To mimic a wildfire situation, for example, prototypes were simultaneously exposed to a toxic gas as well as smoke, generated in situ by burning wood chips. The general setup and a photograph of it are provided in a and b in FIG. 7. Exposed to 50 ppm Cl₂ gas followed by two intermittent bursts of dense smoke, device performance was practically unaltered by the smoke, tripping in less than 20 s (c in FIG. 7). Since smoke produces carbon NPs, as those isolated from the smoke generated (see luminescent carbon dots isolated from the smoke in d in FIG. 7), these nanoparticulates appear to not interfere with the device functioning as intended.

### COCl₂ Gas Sensors: Testing and Sensor Refinements

As outlined above, this approach rests on the generation of a sensor platform that is ultimately capable of simultaneously detecting multiple toxic gases. While some use cases could be specific to just one gas, for example Cl₂ gas in water treatment plants, others would require the detection of mixtures of toxic gases. To highlight an additional example, new prototypes for detection of phosgene (COCl₂) were examined. Like Cl₂ gas, COCl₂ is a choking agent that harms unprotected individuals mainly in the respiratory tract, i.e., in the nose, throat, and particularly, the lungs. Death can result by asphyxia if membranes swell. COCl₂ destroys lung tissues through a complicated sequence of events. As an insidious poison (toxic dose: ≥30 ppm min⁻¹), COCl₂ is very hazardous owing to an unpredictable asymptomatic latent phase that precedes the onset of life-threatening pulmonary edema. The gas poses significant risks for firefighters in the vicinity of fires that involves it as a combustion product of Freon (often a mixture of chlorofluorocarbons or CFCs and hydrofluorocarbons) refrigeration equipment or leaks, in gasoline fires, or while fighting fires using chlorine-based halons or halotrons, which are liquid streaming or gaseous flooding agents used to prevent the spread of fires. Hence, firefighters are a group with particularly high-risk factors.

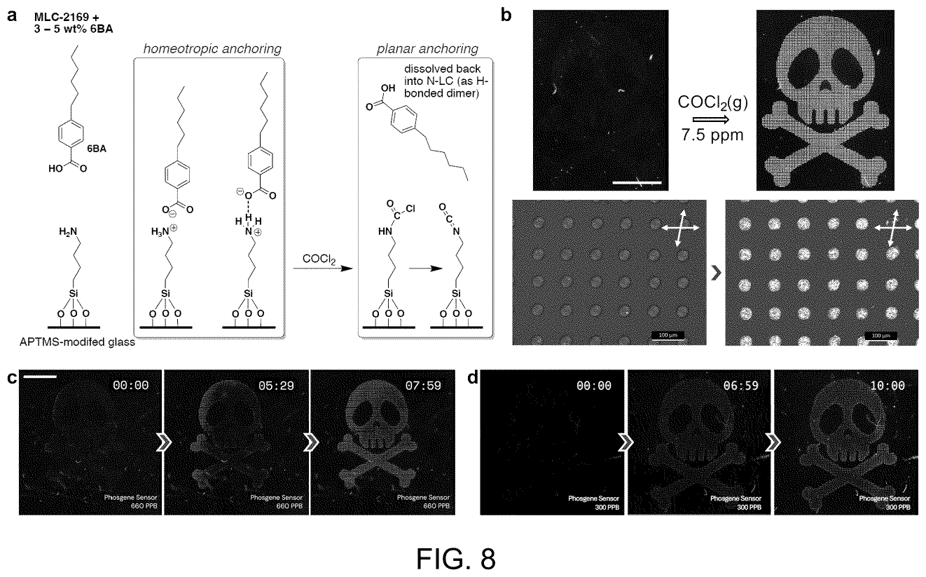

To fabricate more effective, zero-power COCl₂ sensors while further simplifying the process and making eventual production even more economical, the glass substrate may be functionalized with a siloxane-based self-assembled monolayer (SAM). The selected (3-aminopropyl) trialkylsilane (APTES or APTMS; ethyl or methyl) coated glass slides are commercially available or can be generated in the lab. It should be recognized that other amino group-terminated silanes could alternatively be used. Doping the chemically inert N-LC with a mesogenic carboxylic acid such as 6 BA (4-hexylbenzoic acid) in situ generates homeotropic anchoring by a combination of hydrogen bonding and ionic interactions, which is then disturbed by the reaction of the amine group with COCl₂. 6 BA then diffuses back into the N-LC just as the disulfides did that were generated by the cleavage of DDT from the GNP surface after reaction with Cl₂ gas (a in FIG. 8). A prototype of such a device in action is shown in b in FIG. 8. At a concentration of COCl₂ of 7.5 ppm, this sensor started to trip after just 20 s, with the full change in light transmission after 150 s. Further optimization of prototypes (lower drop volume, concentration of 6 BA, etc.), allowed detection of even sub-ppm levels of COCl₂ (down to 0.1 ppm; lower than most regulatory limits listed and lower than NIOSH's Immediately Dangerous to Life or Health (IDLH) limit set at 2 ppm that can be detected in less than 2 minutes (c and d in FIG. 8).

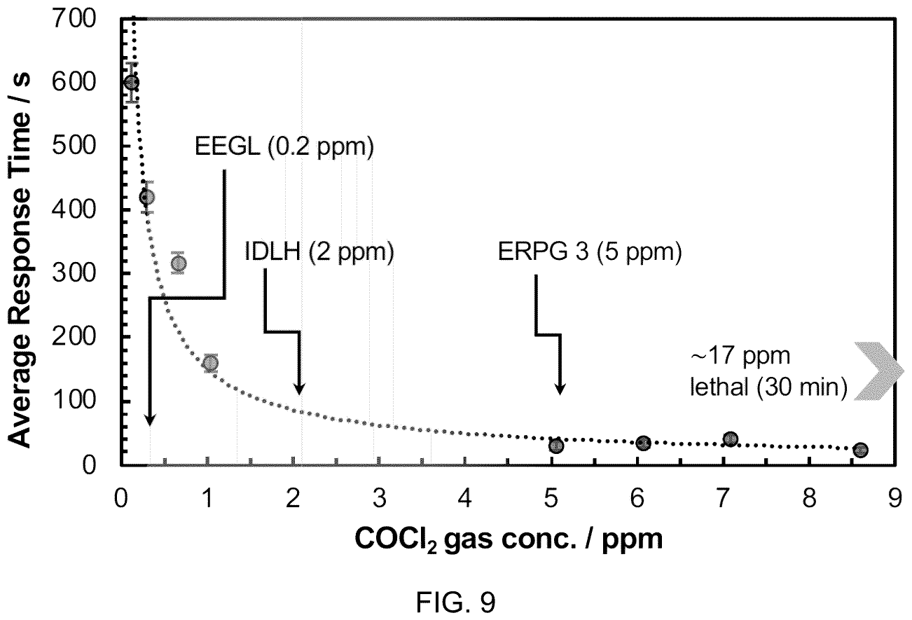

A plot of the sensor prototype response time vs. the concentration of COCl₂ (FIG. 9) then shows the same, and as expected for a diffusivity-controlled process, exponential decay seen already for the Cl₂ sensors. The graph further highlights relevant levels set by OSHA and NIOSH, and thus, demonstrates that the new prototypes operate at desirable response times and sensitivity levels. Levels of COCl₂ well below the ERPG 3 (FIG. 9) or the 30-min lethal limit can now be detected in just a few seconds.

### Discussion

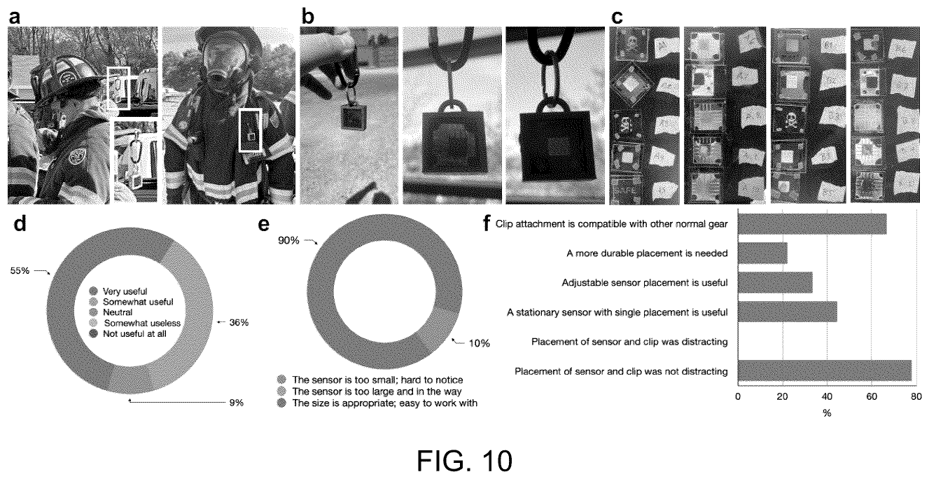

While the advantages of the new sensor platform design with respect to response time and sensitivity are encouraging, another key aspect is how potential users judge the operation, form factor, integration with existing gear, and potential placement of these devices. To do so, firefighters conducted several training tests. Prototype devices of the sensors differing in design, packaged in special 3-D printed casings, were affixed with carabiners to various spots on their turnout gear or on the helmets (a-c in FIG. 10). After wearing the devices during their training exercise, firefighters were asked to comment on the usefulness, form factor, size, and visibility as well as to provide feedback that will educate further prototype improvements (d-f in FIG. 10). One of the key lessons learned here was that a more durable placement of such devices would be desired and that stationary sensors for fire stations or fire trucks would be highly advantageous for firefighters.

Given what have learned about this next generation of LC-based zero-power toxic gas sensors, several improvements in sensitivity, speed of response, and packaging are already in motion for the next stages of development. For example, COCl₂ sensors are much faster than the Cl₂ gas sensors. Both sensor prototypes rely on the diffusivity of the gas through the N-LC droplets and on the dissolution of a released molecular species back into the N-LC. The key difference is that the reaction of Cl₂ gas with the DDT-GNPs occurs at the NP-ligand interface and not at the ligand periphery as in the case of the APTMS-coated glass slides in the case of the COCl₂ sensors. Thus, the current focus for multiple other toxic gases and vapors is on that specific periphery closest to the N-LC. Further improvements to the response time and sensitivity of the Cl₂ gas but also for other types of gases/vapors are currently relying on promesogenic (pro-N-LC)-type ligands on the GNP, glass or even polymer interfaces that would much faster redissolve back into the N-LC after release during the chemical reaction with the toxic gas or vapor. To solve the issue with water infiltration, Applicant is already planning on coating the devices and/or casing with superomniphobic materials to prevent water infiltration. Moreover, larger sensors for stationary placement as well as better solutions for affixing the devices to turnout gear are currently in the works.

### CONCLUSIONS

The aim of this work was to fully re-engineer the design of Applicant's previously disclosed zero-power liquid crystal-based toxic gas and vapor sensors, especially given earlier limitations with respect to the response time. While ideal for dose×time sensor devices, the prior design's use for immediate exposure was hampered by the slow diffusion of the target toxic gases through the N-LC sublayer to reach the reactive GNP patterns that trigger the anchoring transition and generate the ensuing image, pattern, or text.

The current design, described here, drastically minimizes this issue by providing direct access for the toxic gas or vapor to ink-jet printed N-LC droplets printed (patterned) atop the reactive species, either surface-functionalized GNPs or silane-monolayers. What used to be mm² of N-LC that the gas needed to diffuse through are now only ˜500 nm. This, in turn, leads to sensor response times of just seconds for concentrations in the higher ppm range (>5 ppm for COCl₂ and >30 ppm for Cl₂) or a few minutes for the lower ppm and even ppb range. The response time exponentially decreases with increasing gas concentrations and can be further tuned (reduced) by various device manufacturing parameters.

Interference and field-testing experiments further revealed the suitability of these devices for real world applications and in harsh environments such as elevated temperatures, high humidity, and smoke.

Finally, the new device design, based on possible combinations of reactive interfaces and doped N-LCs, continues to promise high versatility for a range of other toxic gases and vapors, several of which are already pursued in the laboratory. The new device concept also remains very inexpensive to manufacture, highly suitable for multi-gas sensing on the same device, and appropriate for remote sensing via a possible combination with a light source and photodetector (e.g., on drones or affixed to robotic vehicles).

The following examples are provided to illustrate the systems and methods of the present disclosure. The examples are merely illustrative and are not intended to limit the disclosure to the materials, conditions, or process parameters set forth therein.

### EXAMPLES

### Experimental Section/Methods

Materials: Tetraoctylammonium bromide (TOAB), n-dodecanethiol, hydrogen tetrachloroaurate, and sodium borohydride were purchased from Sigma-Aldrich. All solvents used were HPLC grade and dried using standard procedures. All glassware for synthesis is cleaned with aqua regia. Deionized water (18.0 MΩ cm, Barnstead E-pure ultrapure water system) was used during all synthetic steps. The nematic liquid crystal MLC-2169 (phase transitions: −60 N 110 Iso, [° C.]) was provided by Merck KGaA; 5 CB (phase transitions: Cr 25 N 35 Iso [° C.]) and 4-hexylbenzoic acid (6 BA; phase transitions: Cr 96 N 113 Iso [° C.]) were purchased from Sigma Aldrich and Thermo Fisher Scientific, respectively. Calibrated chlorine and phosgene gases balanced with nitrogen at various ppm levels were obtained from

MESA Specialty Gases & Equipment.

Materials Characterization: ¹H NMR spectra were obtained in CDCl₃ at ambient temperature on a Bruker DMX 400 MHz spectrometer and referenced internally to residual solvent peaks at 7.26 (¹H). Visible light absorption spectra of the NP in chloroform were recorded using a Thermo Scientific Evolution 220 or an OLIS 14 UV/Vis/NIR spectrophotometer.

Transmission electron microscopy (TEM) analysis of GNP size and shape was accomplished using an FEI Tecnai TF20 TEM at an accelerating voltage of 200 kV. Samples were prepared by evaporating a drop of dilute chloroform-nanoparticle solution onto a carbon-coated copper 400 mesh TEM grid and dried. Scanning electron microscopy (SEM) analysis was performed using a Quanta 450 FEG SEM with or without prior metal deposition. TGA analysis was accomplished using a TA Instruments Q500 TGA. Between 5-10 mg of dried NP was placed on a clean platinum weighting pan and heated from 25-500° C. at a ramping rate of 10° C. min⁻¹. Material viscosity was measured using an Anton Paar ViscoQC 300-L viscometer.

Contact angle measurements were done using a Rame-Hart 250 goniometer at 20° C. Assembled sensor services were observed using an Olympus BX-53 polarized light optical microscope and photos recorded with a QImaging GO-3 CMOS camera.

Printing: Nanoparticle ink is prepared by dispersing dried nanoparticles in o-xylene to produce a concentration of 45 mg ml⁻¹. The dispersion is filtered through a 0.2-μm PTFE filter before injection into a Dimatix ink-jet cartridge per the manufacturer's instructions. A desktop material ink-jet printer by Fujifilm Dimatix DMP-2800 and compatible cartridges DMCLCP-11610 and Fujifilm Dimatix® Samba. Waveforms needed for the 16-jet DMCLCP-11610 cartridges are different from the 12-jet Samba cartridges. The 16-jet DMCLCP-11610 jets 10 pL of fluid and is optimized for fluids with properties to include a viscosity of 10-12 cP and surface tension 28-30 dynes cm⁻¹. The 12-jet Samba jets 2.4 pL of fluid and is optimized for fluids with properties to include a viscosity of 4-8 cps and surface tension 28-32 dynes cm⁻¹. DDT-GNP ink at 45 mg ml⁻¹ has a dynamic viscosity of 10 cP and is jetted at the lowest accelerating voltage without the formation of satellite droplets of 20-25 V per the waveform. The N-LC MLC-2169 or 5 CB was degassed and injected into a 12-jet Samba cartridge per the manufacturer's instructions. Viscosity of the N-LC is optimal for the Samba cartridge at 65° C., 10.3 cP, and is jetted without the formation of satellite droplets at an accelerating voltage of 25-30 V per the waveform.

Cleaning clogged cartridge nozzles is carried out through both the printer cleaning programs Spit, Blog, and Purge or manually by blotting nozzles against lint-free cleanroom wipes saturated with the ink's solvent.

Images of the desired sensor motif or design are generated as binary colored bitmap files and produced into inkjet patterns through the Dimatix programming, converting each pixel of the pattern to represent a drop of printed fluid. Nanoparticle ink is printed onto a glass treated with a deposited alignment layer of polyimide or SiOₓ. The plate is then dried at 45° C. under vacuum for over 24 h to remove ink solvent. Once dry, the plate is placed again on the printer and the same printing process is executed with the N-LC as detailed above.

Device Assembly: UV-sensitive adhesive Norland 68 (NO68 is used to fix spacers between the glass of the sensor and a protective glass cover over the sensor area to preserve the LC structure and patterning. Silica spacers of 5, 10, or 20 μm are mixed with NO68 at 5 wt % to generate a small gap or color-coded Travers Tool Co. plastic shims are glued using NO68 for larger gaps, 101.6-762 μm. The adhesive is cured for over 8 min with an Electro-Lite ELC4000 UV curing unit while the cell is placed under plastic cover and light vacuum.

Assembled cells are sealed in a plastic casing after vacuum sealing to preserve their integrity.

Toxic Gas Testing: Testing of the sensor prototypes in the presence of the toxic gases was accomplished utilizing the setup shown in FIG. 3. The sealed glass testing chamber with two outlets was set between a crossed polarizer and analyzer. A TOMLOV DM602 recording microscope is used to record video of the sensor during testing, lit by a backlight.

Two Azbil Corporation digital mass flow controllers (MFC), MPC0005BBRN010000, are utilized to adjust the flow (L min⁻¹) of compressed air passed through a carbon Omnipure CL6ROT33-B filter and compressed calibrated gas balanced with nitrogen, MESA Specialty Gases & Equipment, labeled MFC2 and MFC1 respectively. A T-joint connects both MFC output lines, diluting the calibrated chlorine gas to a desired ppm concentration. Total gas flow is maintained at 3 L min⁻¹ between a combined MFC1 and MFC2.

$${C}_{gas} = \frac{{R}_{gas}}{{R}_{gas} + {R}_{air}} {C}_{0} \qquad ( 1 )$$

Using Equation (1) to adjust the MFCs' flow rate R of both air and calibrated gas, a wide range of gas concentrations, C_{gas}, can be generated from the original chlorine concentration Co. Diluted toxic gas is directed through a neutralizing system composed of activated carbon after the ventilation line and soda as well as activated carbon after the testing chamber before being exhausted. A switch allows the diversion of the toxic gas directly to the neutralizing system or to the testing chamber. Once the line to the compressed toxic gas is open, both MFCs are allowed to stabilize before diverting the diluted gas into the testing chamber. This assures that the desired ppm of toxic gas is present immediately after the line to the testing chamber is opened. The testing chamber contains a volume of 0.56 L which is assumed to be filled with a uniform dispersion of chosen ppm of toxic gas at t=11 s after diverting gas to the chamber. After sensing, the compressed toxic gas line is closed, and air is allowed to evacuate the toxic gas from the chamber for at least 1 min before the chamber is deemed safe to open.

Smoke Interference Testing: Testing sensor reaction in the presence of Cl₂ gas with the interference of smoke is accomplished with the system as seen in FIG. 7. The sealed glass testing chamber with three outlets is set between a crossed polarizer and analyzer. A TOMLOV DM602 recording microscope is used to record video of the sensor during testing, lit by a backlight. Two Azbil Corporation Digital Mass Flow Controllers (MFC), MPC0005BBRN010000, are utilized to adjust the flow (L min⁻¹) of compressed air passed through a carbon Omnipure CL6ROT33-B filter and compressed calibrated 1,015 ppm chlorine gas balanced with nitrogen, MESA Specialty Gases & Equipment. A Y-joint passes air diluent through a quartz tube that holds non-flammable glass wool with commercially available smoking pellets. Heat is applied to the quartz tube to generate smoke from the smoking pellets. A pinch point is utilized to control the amount of air supplied to burning pellets as well as control the quantity of addition of smoke qualitatively. A combined Y- and T-joint allow for the dilution of original concentration of calibrated Cl₂ gas with air or air and smoke to reach a desired concentration of gas. Total flow is maintained at 3 L min⁻¹ between a combined MFC1 and MFC2.

### H₂S Sensors Utilizing Printed Nematic Liquid Crystal Patterns on Metal-Salt Based Reactive Substrates

H₂S exposure is a serious concern due to its high toxicity, environmental presence, and explosive potential. This hazardous gas is naturally generated through geological processes, making it a significant risk in industries such as natural gas extraction, mining, and oil production. While its dangers are well known in the gas and mineral extraction industries, they extend far beyond these fields. H₂S is also a common byproduct in paper manufacturing, textile processing, and agricultural operations. For firefighters, the threat is a frequent occurrence since burning sulfur-containing materials, such as rubbers and plastics, can quickly produce harmful concentrations of the gas.

Although H₂S is explosive at concentrations of 43,000 ppm, its extreme toxicity poses a far greater threat. The gas becomes an irritant at just 2-5 ppm, prompting NIOSH to set a 10-minute recommended exposure limit (REL) of only 10 ppm. Even slight increases above this limit can cause significant adverse health effects. At 20 ppm, symptoms such as headaches, dizziness, and impaired memory may occur. At 100 ppm, H₂S can rapidly cause loss of smell, drowsiness, and, with prolonged exposure, even death. Given these risks, continuous monitoring is essential for workers in environments where exposure is possible, yet current detection methods remain insufficient in certain settings.

Battery-operated devices are available to detect H₂S gas. They are often bulky, expensive, and cumbersome to use. As a result, monitoring every worker in a potential exposure environment is often impractical. Smaller, more affordable, and simpler alternatives exist in the form of powerless detectors. These include chemically treated papers, badges, and colorimetric tubes. While useful in some settings, these methods rely on detecting subtle color changes, introducing a high degree of subjectivity when identifying potentially life-threatening concentrations. Many of these solutions are designed to measure cumulative exposure over days or weeks rather than detect acute, high-level exposures. This means workers may remain vulnerable during dangerous, immediate exposures.

Recent research has explored innovative methods for developing sensitive, powerless H₂S sensors. One approach involves using chemically treated papers with treated with inorganic salts or dyes. These alternatives have demonstrated low detection limits, fast response times, and simple manufacturing requirements, but still rely on subjective color changes to show detection. Another color changing approach uses colorimetric solutions based on reactions involving nanoparticles. Such gold nanoparticle-based sensors exhibit color shifts upon aggregation. By designing reactions that trigger such nanoparticle aggregation, highly sensitive H₂S detection solutions can be achieved. However, such paper-based systems have not yet been demonstrated in finished devices. Furthermore, due to the complexity and cost of nanoparticle solutions, using them in bulk quantities for sensing remains impractical compared to more cost-effective methods.

In contrast, LC-based sensing approaches can offer viable alternatives to other zero-power detection methods. It has been extensively studied for toxic gas sensing and has demonstrated potential for detecting a wide range of gases. Several studies have explored its application for H₂S detection. One approach utilized electronic measurements on discotic LC thin films, achieving good sensitivity. Another study employed a fully powerless method, leveraging interactions between the nitrile group of the N-LC and lead perchlorate salt. This design successfully detected concentrations as low as 1 ppm of H₂S across a wide range of temperatures and humidity. However, it exhibited relatively long response times compared to other sensing techniques and required multiple complex fabrication steps, including gold monolayer deposition and micropillar fabrication. While the engineering behind the device was complex, its use of a common perchlorate salt shows strong potential for rapid sensing. Perchlorate and tetrafluoroborate salts have been widely studied in LC-based sensing for gases beyond hydrogen sulfide. This is because nitrile groups that are commonly found in LC molecules readily bind to these salts, inducing homeotropic alignment. However, this approach is limited in two important ways. First, it relies exclusively on perchlorate and tetrafluoroborate salts, which are not always available in combination with different metal cations. A sensing mechanism based on more common anion-containing salts, such as acetates or nitrates, could offer a simpler and less expensive solution. Second, it depends heavily on nitrile groups within the liquid crystal. To induce the homeotropic alignment needed for sensing, a large percentage of the mixture must contain nitrile groups. By finding interactions that work with other functional groups a more diverse sensing system could be developed, which would further allow for better control over interferences, response times, and sensitivities.

A new approach to metal-salt reactive surfaces in LC sensing by using the interaction between a carboxylic acid-containing N-LC and lead (II) cations from common lead salts is described. Printing droplets of a mixture of 3 wt % 6 BA in 5 CB onto substrates coated with lead (II) acetate or lead (II) nitrate induced homeotropic alignment in the nematic droplets. This alignment was not seen in samples containing only 5 CB. Without wishing to be bound by theory, it is believed that this alignment is caused by the attraction between the lead (II) cation and the carboxylic acid groups of 6 BA. This interaction appears to mirror the nitrile-perchlorate attraction seen in other sensors, but by using a carboxylic acid instead of a nitrile group, thereby opening the door to a much broader range of reactive salts. While the mixture still contains nitrile groups, there was no evidence that they were responsible for inducing the experimentally observed homeotropic alignment. When using fluorinated N-LC mixtures such as 3 wt % 6 BA in TL205, the sensors still showed homeotropic alignment when printed on lead salt-coated surfaces. For the sensor to function, the alignment must shift from a dark homeotropic state to a bright planar state between crossed polarizers upon exposure to target gas. This means that the sensing mechanism depends on lead (II) binding to the carboxylic acid groups of 6 BA that is altered to lead (II) sulfide upon exposure to hydrogen sulfide. When exposed to H₂S, the sensors transition rapidly from a homeotropic to planar state, which confirms that the lead acetate and hydrogen sulfide detection mechanism can be directly applied to a liquid crystal platform. It also suggests that not only the cation, but that the anion also influences the carboxylic acid's binding affinity as well. This is similar to the role of perchlorate in nitrile-based systems. While the exact reason this happens is still unclear, prior work suggests that electron affinity may play a significant role in functional groups binding to various salts.

The final new sensors demonstrate a wide detection range from about 1 to 100 ppm, with response times typically measured in seconds. At 100 ppm, a level considered deadly after 48 h of exposure, the sensor responded in just 4 seconds. Decreasing this concentration to 10 ppm, the NIOSH 10-minute REL, sensors responded within 20 seconds. Decreasing it even further to 990 ppb, a concentration beyond harmful limits, the device detected the gas in just about 4 minutes. These results show that the design has significant potential to alert users of these sensors well before serious harm can occur. Compared to previous H₂S LC-based sensors, the printed design shows some clear advantages. For example, the earlier design using lead (II) perchlorate and micropillars had similar detection limits (1 to 20 ppm) but significantly slower response times. At 20 ppm, the micropillar design had a lag time of around 3 minutes before indicating the presence of H₂S, while our printed design responds in just 13 seconds. This improvement is believed to come from two main design choices. First, the printed N-LC droplets allow for improved gas flow through the device and reduced diffusion distance to the reactive surface species, compared to the more enclosed micropillar geometry. Second, lead (II) acetate may react more quickly with H₂S than lead (II) perchlorate.

### Materials

The N-LCs 4-cyano-4′-pentylbiphenyl (5 CB), exhibiting thermotropic phase transitions (Cr 25 N 35 Iso [° C.]), and 4-hexylbenzoic acid (6 BA), characterized by phase transitions (Cr 96 N 113 Iso [° C.]), were procured from Synthon and Thermo Fisher Scientific, respectively. A certified gas mixture containing H₂S at a calibrated concentration of 250 parts per million (ppm), balanced with ultra-high-purity nitrogen, was sourced from MESA Specialty Gases & Equipment. Lead (II) acetate trihydrate (CAS No. 6080-56-4), utilized as the metal salt precursor for film deposition, was obtained from Sigma-Aldrich. All aqueous solutions were prepared using ultrapure deionized water (resistivity ≥18.0 MΩ·cm), obtained from a Barnstead E-pure purification system. Ethanol of analytical grade, employed as a component in the solvent system, was also acquired from Sigma-Aldrich.

### Substrate Cleaning and Film Deposition

Glass substrates intended for the construction of hydrogen sulfide gas sensors underwent a stringent and methodically structured multistep cleaning protocol within a controlled cleanroom environment to ensure the comprehensive removal of surface-adhered contaminants and to facilitate uniform precursor film adhesion. The decontamination sequence commenced with sequential ultrasonication in a laboratory-grade soap mixture, deionized water (resistivity ≥18.2 MΩ·cm) and subsequently in analytical-grade isopropanol, each for a duration of 15 min, effectively dislodging both hydrophilic and hydrophobic impurities. Following this, substrates were subjected to oven drying, accompanied by nitrogen-assisted surface cleaning to eliminate residual moisture and particulates. To further augment surface wettability and eradicate trace organic residues, a 15 min oxygen plasma treatment was performed under low-pressure conditions. Immediately thereafter, a precursor formulation comprising 10 mmol of lead (II) acetate trihydrate dissolved in a binary solvent mixture of ethanol and deionized water in a volumetric ratio of 70:30 was uniformly dispensed onto the prepared substrates and deposited via spin coating at 1500 rpm for 30 seconds, yielding a conformal and homogeneous film. The deposited films were subsequently soft-baked at 80° C. for 10 minutes to facilitate controlled solvent evaporation, initiate partial crystallization, and enhance film stability.

### Liquid Crystal Printing

A mixture of 3% 6 BA and 97% 5 CB was filtered through a 0.2 μm PTFE filter as it was injected into a Dimatix Samba Materials Cartridge and loaded into a Fujifilm Dimatix DMP-2800 printer. The material was printed using the standard Samba cartridge waveform provided with the Dimatix Drop Manager software. The N-LC mixture was heated to 55° C. and printed between 30-35 V. The voltage was adjusted until uniform droplets formed. All prints were made with a drop height of 600 μm, a meniscus setpoint of 0.5 inches H₂O, and the bed temperature at room temperature. After printing, substrates were stored under nitrogen at room temperature until testing.

### Toxic Gas Testing

Testing of the sensor prototypes in the presence of the toxic gases was accomplished utilizing the setup described earlier. The sealed glass testing chamber with two outlets was set between a crossed polarizer and an analyzer. A TOMLOV DM602 recording microscope was used to record video of the sensor during testing, lit by a backlight. Two Azbil Corporation digital mass flow controllers (MFC), MPC0005BBRN010000, were utilized to adjust the flow (L·min⁻¹) of compressed air passed through a carbon Omnipure CL6ROT33-B filter and 250 ppm H₂S calibrated gas cylinder balanced with nitrogen, MESA Specialty Gases& Equipment. A T-joint connects both MFC output lines, diluting the calibrated H₂S gas to a desired ppm concentration. Total gas flow was maintained at 2 L·min⁻¹ between the combined MFCs. Diluted H₂S gas was directed through a neutralizing system composed of activated carbon after the ventilation line as well as activated carbon after the testing chamber before being exhausted. A switch allows the diversion of the H₂S gas directly to the neutralizing system or to the testing chamber. Once the line to the compressed toxic gas was open, both MFCs were allowed to stabilize before diverting the diluted gas into the testing chamber. This assures that the desired ppm of toxic gas was present immediately after the line to the testing chamber was opened. The testing chamber contains a volume of 0.56 L which was assumed to be filled with a uniform dispersion of chosen ppm of toxic gas at t=17 s after diverting gas to the chamber. After sensing, the compressed toxic gas line was closed, and air was allowed to evacuate the toxic gas from the chamber for at least 1 min before the chamber was deemed safe to open.

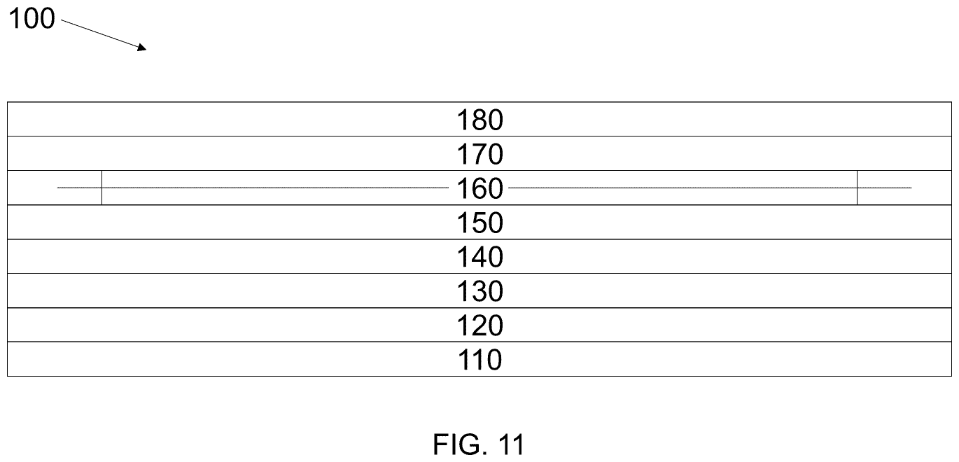

FIG. 11 is a cross-sectional view of a non-limiting embodiment of a sensor 100 in accordance with some embodiments of the present disclosure. The sensor 100 includes, in sequence, a first polarizer 110, a first substrate 120, an alignment underlayer 130, printed nanoparticles or SAMs 140, a patterned liquid crystal layer 150, spacers 160, a second substrate 170, and a second polarizer 180.

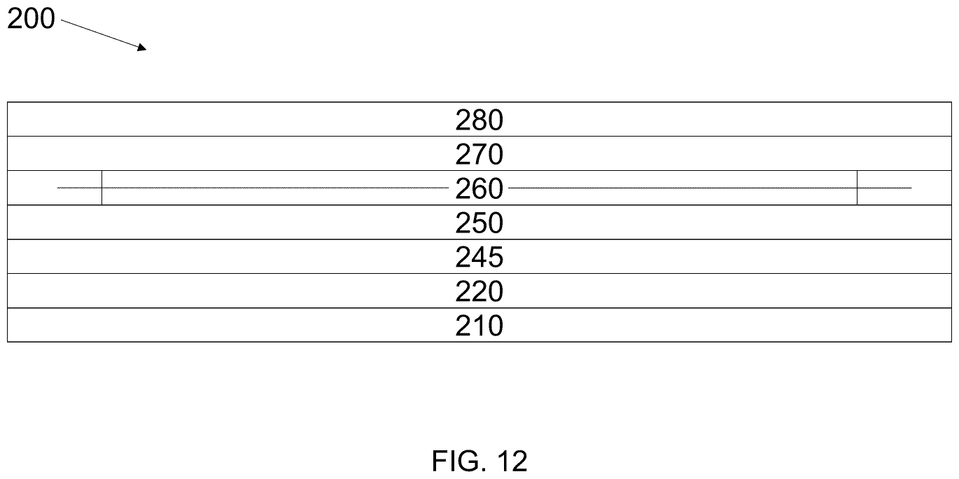

FIG. 12 is a cross-sectional view of a non-limiting embodiment of a sensor 200 in accordance with some embodiments of the present disclosure. The sensor 200 includes, in sequence, a first polarizer 210, a first substrate 220, a metal salt layer 245, a patterned liquid crystal layer 250, spacers 260, a second substrate 270, and a second polarizer 280.

In some embodiments, the metal salt includes a metal acetate or a metal nitrate.

The metal may be lead. Other metals include lithium, sodium, potassium, rubidium, cesium, francium, beryllium, magnesium, calcium, strontium, barium, radium, lanthanum, cerium, praseodymium, neodymium, promethium, samarium, europium, gadolinium, terbium, dysprosium, holmium, erbium, thulium, ytterbium, lutetium, actinium, thorium, protactinium, uranium, neptunium, plutonium, americium, curium, berkelium, californium, einsteinium, fermium, mendelevium, nobelium, lawrencium, scandium, yttrium, titanium, zirconium, hafnium, rutherfordium, vanadium, niobium, tantalum, dubnium, chromium, molybdenum, tungsten, seaborgium, manganese, technetium, rhenium, bohrium, iron, ruthenium, osmium, hassium, cobalt, rhodium, iridium, meitnerium, nickel, palladium, platinum, darmstadtium, copper, silver, gold, roentgenium, zinc, cadmium, mercury, copernicium, aluminum, gallium, indium, thallium, nihonium, tin, flerovium, bismuth, moscovium, polonium, and livermorium.

In some embodiments, the metal is selected from alkali metals, alkaline earth metals, lanthanides, actinides, transition metals, and post-transition metals.

All documents mentioned herein are hereby incorporated by reference for the purpose of disclosing and describing the particular materials and methodologies for which the document was cited.

The exemplary embodiment has been described with reference to the preferred embodiments. Obviously, modifications and alterations will occur to others upon reading and understanding the preceding detailed description. It is intended that the exemplary embodiment be construed as including all such modifications and alterations insofar as they come within the scope of the appended claims or the equivalents thereof.

What is claimed:

1. A sensor comprising, in sequence:
a first polarizer having a first polarization direction;
a first transparent substrate;
optionally an alignment underlayer;
a reactive layer comprising:
nanoparticles substantially covered by one or more ligands capable of selectively, chemically reacting with one or more gases; or
a self-assembled monolayer capable of selectively, chemically reacting with one or more gases;
an inert nematic liquid crystal layer patterned on the reactive layer;
a second transparent substrate; and
a second polarizer having a second polarization direction perpendicular to the first polarization direction.

2. The sensor of claim 1, wherein the sensor comprises the alignment underlayer; and wherein the alignment underlayer comprises polyimide or SiOₓ.

3. The sensor of claim 1, wherein the nanoparticles comprise metallic nanoparticles.

4. The sensor of claim 1, wherein the metallic nanoparticles comprise gold, silver, platinum or palladium.

5. The sensor of claim 1, wherein the reactive layer comprises aliphatic thiol-capped gold nanoparticles.

6. The sensor of claim 1, wherein the one or more gases comprise chlorine (Cl₂) gas.

7. The sensor of claim 1, wherein the reactive layer comprises a siloxane-based self-assembled monolayer.

8. The sensor of claim 1, wherein the reactive layer comprises an amino group-terminated silane.

9. The sensor of claim 1, wherein the reactive layer comprises a (3-aminopropyl) trialkylsilane.

10. The sensor of claim 9, wherein the (3-amonopropyl) trialkylsilane comprises (3-aminopropyl) triethylsilane or (3-aminopropyl)trimethylsilane.

11. The sensor of claim 1, wherein the inert nematic liquid crystal layer is doped with a carboxylic acid that is miscible with the nematic liquid crystal and induces homeotropic anchoring.

12. The sensor of claim 1, wherein the inert nematic liquid crystal layer is doped with an aliphatic carboxylic acid having an aliphatic chain length of C₃ to C₁₂.

13. The sensor of claim 1, wherein the inert nematic liquid crystal layer is doped with a mesogenic carboxylic acid.

14. The sensor of claim 1, wherein the inert nematic liquid crystal layer is doped with trans-4-hexylcyclohexane carboxylic acid and/or 4-hexylbenzoic acid).

15. The sensor of claim 1, wherein the sensor does not include the alignment underlayer.

16. The sensor of claim 1, wherein the one or more gases comprises phosgene (COCl₂).

17. The sensor of claim 1, wherein the inert liquid crystal layer comprises patterned droplets.

18. The sensor of claim 17, wherein the droplets have volumes in a range of from about 0.1 pL to about 100 pL and center-to-center distances between adjacent droplets are in a range of from about 20 μm to about 250 μm.

19. A method of determining whether a target gas is present in an environment, the method comprising:
providing a sensor to the environment; and
visually inspecting the sensor for a change indicating the target gas is present;
wherein the sensor comprises:
a first polarizer having a first polarization direction;
a first transparent substrate;
optionally an alignment underlayer;
a reactive layer comprising:
nanoparticles substantially covered by one or more ligands capable of selectively, chemically reacting with the target gas; or
a self-assembled monolayer capable of selectively, chemically reacting with the target gas;
an inert nematic liquid crystal layer patterned on the reactive layer;
a second transparent substrate; and
a second polarizer having a second polarization direction perpendicular to the first polarization direction.

20. A method of forming a gas sensor, the method comprising:
ink-jet printing nematic liquid crystal droplets onto a reactive species;
wherein the reactive species comprises surface functionalized nanoparticles or silane-monolayers capable of reacting with one or more target gases.
